(19) United States
(12) Patent Application Publication
(10) Pub. No.: US 2024/0115790 A1
(43) Pub. Date: Apr. 11, 2024

(54) DRAINAGE BOTTLE SYSTEM

(71) Applicant: PACIFIC HOSPITAL SUPPLY CO., LTD., Taipei (TW)

(72) Inventors: Jung-Yen TSAI, Taipei (TW); Ming-Chung CHEN, Taipei (TW); Hsuan-Chiao HO, Taipei (TW)

(73) Assignee: PACIFIC HOSPITAL SUPPLY CO., LTD., Taipei (TW)

(21) Appl. No.: 18/376,808

(22) Filed: Oct. 4, 2023

(30) Foreign Application Priority Data
Oct. 5, 2022 (TW) 111137907

(51) Int. Cl. A61M 1/00

(52) U.S. Cl. CPC A61M 1/74; A61M 1/73

(57) ABSTRACT

Disclosed is a drainage bottle system comprising: a system housing, a drainage bottle, a drainage tube, a motorless suction device and a liquid detection device, wherein a piezoelectric suction pump of the motorless suction device generates a negative pressure by means of piezoelectric effect to drive the drainage tube to suck and collect a liquid to be detected into the drainage bottle.

### FIELD OF THE INVENTION

The present invention relates to a chest drainage bottle, and more particularly relates to a drainage bottle system.

### BACKGROUND OF THE INVENTION

A chest drainage bottle is a device for performing a chest drain procedure in cases of pneumothorax, thoracotomy, pleural effusion, empyema, chylothorax, hemothorax, etc. Through the chest drain procedure, it can achieve the functions of draining fluid from the chest cavity to help diagnosis, draining air and blood from the pleural space, and so on.

In a conventional chest drainage bottle, one end of a chest drainage tube is connected to a patient, and the other end of the chest drainage tube is connected to a connection bottle and then connected to a suction device. The suction device is mainly composed of a diaphragm pump and a conventional motor. The motor drives the diaphragm pump to generate a negative pressure to suck fluid from the patient to the connection bottle. However, the motor is noisy and generates electromagnetic interference (EMI), which not only causes discomfort to the patient, but also is not suitable for use in certain medical fields (e.g., MRI room) that may be affected, and therefore there is a need for improvement.

### SUMMARY OF THE INVENTION

Accordingly, one objective of the present invention is to provide a drainage bottle system to solve technical problems in a prior art.

In order to overcome the technical problems in the prior art, the present invention provides an drainage bottle system, comprising: a system housing having a drainage element disposing space; a drainage bottle, mounted on the system housing; a drainage tube, which is disposed within the system housing, having a sucking end extending outside the system housing and a collecting end connected to the drainage bottle through the system housing; a motorless suction device, which is disposed in the drainage element disposing space, including a piezoelectric suction pump and a driving circuit, wherein the piezoelectric suction pump is connected with the drainage tube, the driving circuit is coupled to the piezoelectric suction pump and is configured to drive the piezoelectric suction pump so that the piezoelectric suction pump generates a negative pressure by means of piezoelectric effect to drive the drainage tube to suck a liquid to be detected from the sucking end toward the collecting end, thereby collecting the liquid to be detected via the drainage tube into the drainage bottle; and a liquid detection device, disposed in the drainage element disposing space and corresponding to the drainage bottle to obtain at least one piece of liquid detection information about the liquid to be detected.

In one embodiment of the present invention, the drainage bottle system is provided, wherein the system housing is provided with a front outer surface as a drainage bottle mounting surface on which the drainage bottle is mounted, wherein the collecting end of the drainage tube extends through the drainage element disposing space to the drainage bottle mounting surface to be connected to the drainage bottle.

In one embodiment of the present invention, the drainage bottle system is provided, wherein the motorless suction device further includes a suction tube, one end of the suction tube is connected to the piezoelectric suction pump, the other end of the suction tube extends to the drainage bottle mounting surface to be connected to the drainage bottle, and thereby the piezoelectric suction pump is connected to the drainage tube via the suction tube and the drainage bottle, wherein the piezoelectric suction pump is configured to generate the negative pressure in the drainage bottle so that the liquid to be detected is sucked and collected into the drainage bottle via the drainage tube.

In one embodiment of the present invention, the drainage bottle system is provided, wherein the piezoelectric suction pump is a ceramic piezoelectric suction pump that utilizes mechanical deformation of a piezoelectric ceramic of the ceramic piezoelectric suction pump to generate the negative pressure.

In one embodiment of the present invention, the drainage bottle system is provided, wherein the driving circuit is configured to drive the piezoelectric suction pump with a sinusoidal wave signal or a square wave signal.

In one embodiment of the present invention, the drainage bottle system is provided, wherein the liquid detection device includes at least one of a liquid level sensor, a liquid color sensor and a tag image recognition sensor, wherein the liquid level sensor is used for sensing a liquid level of the liquid to be detected in the drainage bottle to obtain liquid level information as the liquid detection information, the liquid color sensor is used for sensing a liquid color of the liquid to be detected in the drainage bottle to obtain liquid color information as the liquid detection information, and the tag image recognition sensor is used for correspondingly sensing a tag identification code on the drainage bottle to obtain tag identification information as the liquid detection information.

In one embodiment of the present invention, the drainage bottle system is provided further comprising: a cloud transmission device disposed in the drainage element disposing space and coupled to the liquid detection device, wherein the cloud transmission device is configured to transmit the liquid detection information from the liquid detection device to a cloud data analyzing device for cloud data analysis.

In one embodiment of the present invention, the drainage bottle system is provided further comprising: a wireless remote transmission device disposed in the drainage element disposing space and coupled to the liquid detection device, wherein the wireless remote transmission device is configured to wirelessly transmit the liquid detection information from the liquid detection device to a remote mobile device for notification and/or display on the remote mobile device.

In one embodiment of the present invention, the drainage bottle system is provided, wherein the remote mobile device is a wearable mobile device.

With the technical means adopted by the present invention, the drainage bottle system of the present invention replaces the convention structure with the motorless suction device, which utilizes voltage changes to produce repeated mechanical deformations of a piezoelectric element of the piezoelectric suction pump, thereby generating the negative pressure that drives the drainage tube to suck and collect the liquid to be detected into the drainage bottle. In the drainage bottle system of the present invention, the motorless suction device operates with extremely low noise and no electromagnetic interference, which can effectively avoid the discomfort of the patient, and can be applied to various medical fields without worrying about electromagnetic interference, thus effectively solving the technical problems in the prior art.

### BRIEF DESCRIPTION OF THE DRAWINGS

FIG. 1 is a schematic perspective view illustrating a drainage bottle system according to one embodiment of the present invention;

FIG. 2 is a schematic sectional view illustrating the drainage bottle system according to the embodiment of the present invention;

FIG. 3 is a schematic diagram illustrating the principle of operation of a motorless suction device of the drainage bottle system according to the embodiment of the present invention;

FIG. 4 is a schematic diagram illustrating the principle of operation of the motorless suction device of the drainage bottle system according to the embodiment of the present invention; and

FIG. 5 is a schematic diagram illustrating a healthcare system to which the drainage bottle system according to the embodiment of the present invention is applied.

### DETAILED DESCRIPTION OF THE PREFERRED EMBODIMENTS

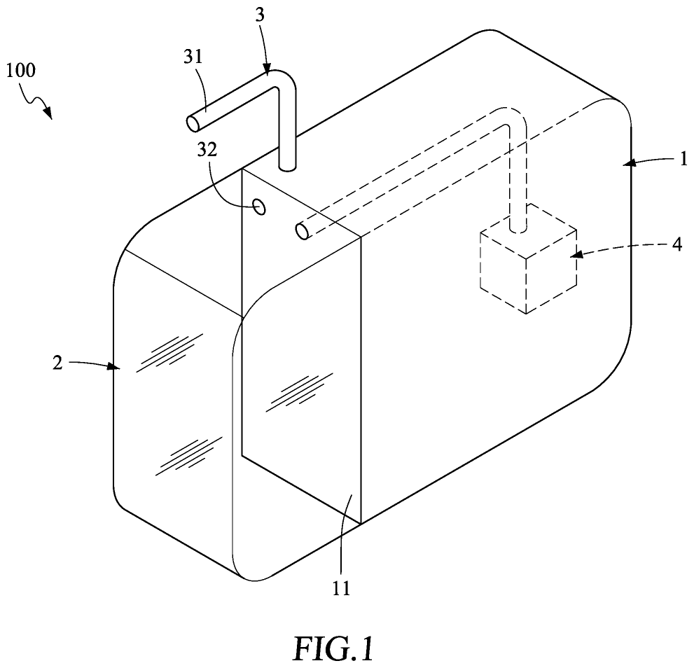

The preferred embodiments of the present invention are described in detail with reference to FIGS. 1 to 5. The description is used for explaining the embodiments of the present invention only, but not for limiting the scope of the claims.

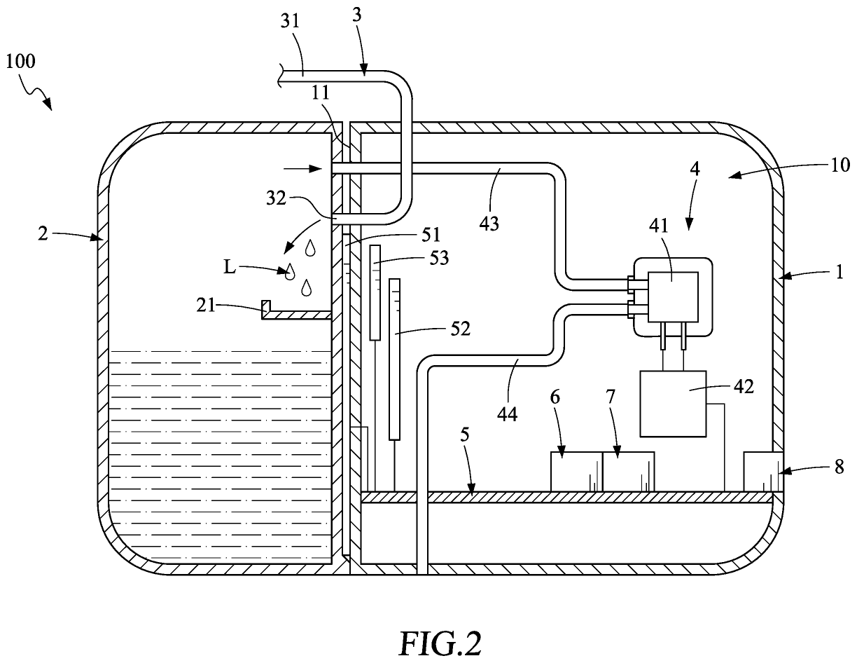

As shown in FIG. 1 and FIG. 2, a drainage bottle system 100 according to one embodiment of the present invention comprises: a system housing 1, a drainage bottle 2, a drainage tube 3, a motorless suction device 4 and a liquid detection device 5.

As shown in FIG. 1 and FIG. 2, a drainage element disposing space 10 is formed inside the system housing 1. The drainage element disposing space 10 is used for the motorless suction device 4 and the liquid detection device 5.

As shown in FIG. 1 and FIG. 2, the drainage bottle 2 is mounted on the system housing 1. The drainage bottle 2 is used to contain fluid drained in a chest drain procedure. Preferably, in order to facilitate visual and instrumental observation of the fluid collected in the drainage bottle 2, the drainage bottle 2 is made of transparent material in this embodiment.

As shown in FIG. 1 and FIG. 2, the drainage tube 3, which is disposed at the system housing 1, has a sucking end 31 extending outside the system housing 1 and a collecting end 32 connected to the drainage bottle 2 through the system housing 1. The sucking end 31 is used for connecting to a patient so that the chest drain procedure can be performed on the patient by using the drainage tube 3.

Specifically, in this embodiment, the system housing 1 is provided with a front outer surface as a drainage bottle mounting surface 11 on which the drainage bottle 2 is mounted, wherein the collecting end 32 of the drainage tube 3 extends through the drainage element disposing space 10 to the drainage bottle mounting surface 11 to be connected to the drainage bottle 2.

As shown in FIG. 1 and FIG. 2, the motorless suction device 4, which is disposed in the drainage element disposing space 10, includes a piezoelectric suction pump 41 and a driving circuit 42, wherein the piezoelectric suction pump 41 is connected with the drainage tube 3, the driving circuit 42 is coupled to the piezoelectric suction pump 41 and is configured to drive the piezoelectric suction pump 41 so that the piezoelectric suction 41 pump generates a negative pressure by means of piezoelectric effect to drive the drainage tube 3 to suck a liquid to be detected L from the sucking end 31 toward the collecting end 32, thereby collecting the liquid to be detected L via the drainage tube 3 into the drainage bottle 2.

Specifically, in this embodiment, the motorless suction device 4 further includes a suction tube 43, one end of the suction tube 43 is connected to the piezoelectric suction pump 41, the other end of the suction tube 43 extends to the drainage bottle mounting surface 11 to be connected to the drainage bottle 2, and thereby the piezoelectric suction pump 41 is connected to the drainage tube 3 via the suction tube 43 and the drainage bottle 2, wherein the piezoelectric suction pump 41 is configured to generate the negative pressure in the drainage bottle 2 so that the liquid to be detected L is sucked and collected into the drainage bottle 2 via the drainage tube 3. Needless to say, the present invention is not limited to this, and the motorless suction device 4 may also utilize other kinds of piping configurations to achieve the function of driving the drainage tube 3 for suction.

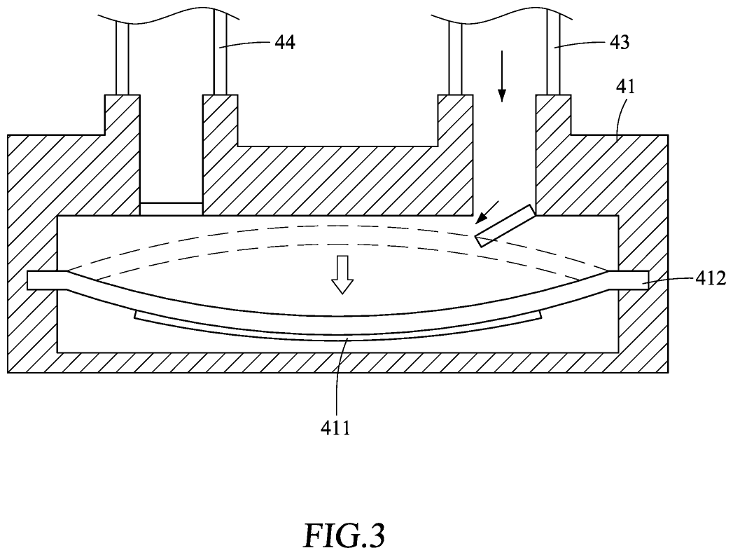

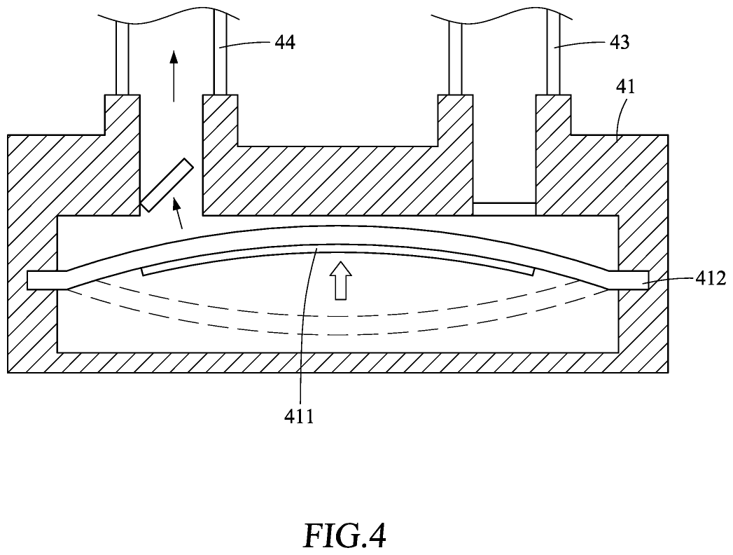

Furthermore, as shown in FIG. 3 and FIG. 4, in this embodiment, the piezoelectric suction pump 41 mainly includes a piezoelectric element 411 and a diaphragm element 412 to which the piezoelectric element 411 is attached. When driven by the driving circuit 42, as shown in FIG. 3, the piezoelectric element 411 stretches to bend the diaphragm element 412 in a downwardly deformed manner, and therefore the volume of an inside space of the piezoelectric suction pump 41 is enlarged so that air in the drainage bottle 2 (FIG. 2) is sucked into the inside space of the piezoelectric suction pump 41 via the suction tube 43, thereby generating the negative pressure in the drainage bottle 2, whereby the liquid to be detected L is sucked and collected into the drainage bottle 2 via the drainage tube 3. Next, as shown in FIG. 4, when the piezoelectric element 411 shrinks to reversely bend the diaphragm element 412 in an upwardly deformed manner, the volume of the inside space of the piezoelectric suction pump 41 is reduced so that the air in the piezoelectric suction pump 41 is squeezed out and exhausted out of the system housing 1 via a exhaust tube 44. Preferably, the piezoelectric suction pump 41 is a ceramic piezoelectric suction pump that utilizes mechanical deformation of a piezoelectric ceramic (i.e., the piezoelectric element 411) of the piezoelectric suction pump to generate the negative pressure. Moreover, in this embodiment, the driving circuit 42 is configured to drive the piezoelectric suction pump 41 with a sinusoidal wave signal or a square wave signal. The sinusoidal wave signal is, for example, a sine wave signal or a cosine wave signal.

As shown in FIG. 2, the liquid detection device 5 is disposed in the drainage element disposing space 10 and corresponding to the drainage bottle 2 to obtain at least one piece of liquid detection information about the liquid to be detected L.

With the above structure, the drainage bottle system 100 of the present invention replaces the convention structure with the motorless suction device 4, which utilizes voltage changes to produce repeated mechanical deformations of the piezoelectric element 411 of the piezoelectric suction pump 41, thereby generate the negative pressure that drives the drainage tube 3 to suck and collect the liquid to be detected L into the drainage bottle 2. In the drainage bottle system 100 of the present invention, the motorless suction device 4 operates with extremely low noise and no electromagnetic interference, which can effectively avoid the discomfort of the patient, and can be applied to various medical fields without worrying about electromagnetic interference, thus effectively solving the technical problems in the prior art.

As shown in FIG. 2, in the drainage bottle system 100 according to one embodiment of the present invention, the liquid detection device 5 includes at least one of a liquid level sensor 51, a liquid color sensor 52 and a tag image recognition sensor 53, wherein the liquid level sensor 51 is used for sensing a liquid level of the liquid to be detected L in the drainage bottle 2 to obtain liquid level information as the liquid detection information, the liquid color sensor 52 is used for sensing a liquid color of the liquid to be detected L in the drainage bottle 2 to obtain liquid color information as the liquid detection information, and the tag image recognition sensor 53 is used for correspondingly sensing a tag identification code on the drainage bottle 2 to obtain tag identification information as the liquid detection information.

Specifically, the liquid level and the liquid color of the liquid to be detected L may be used for diagnosis, and by providing the liquid level sensor 51 and the liquid color sensor 52, it is convenient to obtain the information immediately after the chest drain procedure. In addition, a sampling area 21 may be further provided within the drainage bottle 2. When the liquid to be detected L is sucked into the drainage bottle 2, it will first flow into the sampling area 21 to facilitate inspection and sampling by a healthcare staff. When the liquid in sampling area 21 exceeds a design height, it will flow in the drainage bottle 2. Furthermore, the drainage bottle 2 is preferably mounted on the system housing 1 in a removable manner to facilitate replacement with a new drainage bottle for each chest drain procedure. Moreover, each drainage bottle may be provided thereon with an identification tag, and by sensing the tag identification code of the identification tag by the tag image recognition sensor 53, information (i.e., the tag identification information) about the drainage bottle and/or the chest drain procedure can be quickly obtained. In addition, in this embodiment, the liquid level sensor 51 may be a capacitive liquid level sensor, a liquid level image sensor or a sensor that sensing the liquid level in any other known manner. Similarly, the liquid color sensor 52 and the tag image recognition sensor 53 can each be various types of sensors that sense in any known manner, and the present invention is not limited thereto.

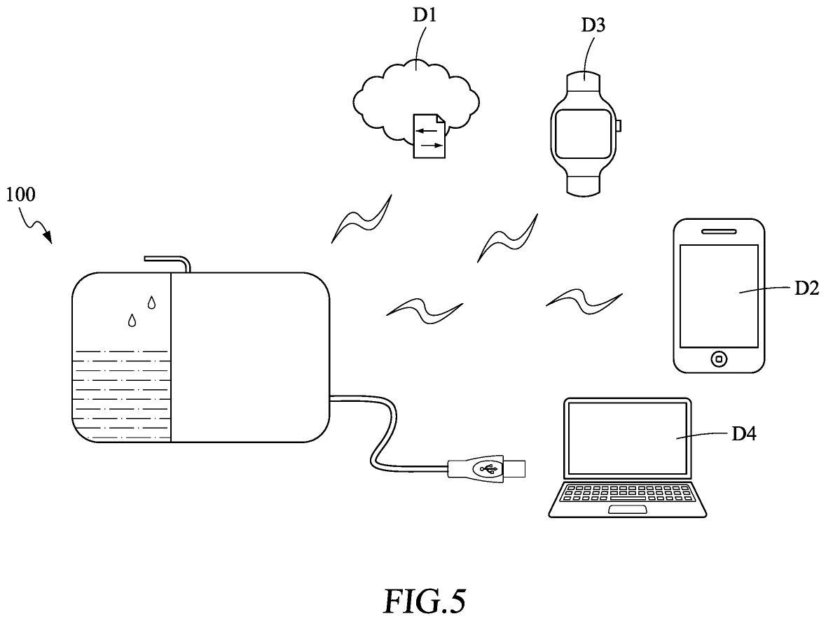

As shown in FIG. 2 and FIG. 5, according to one embodiment of the present invention, the drainage bottle system 100 further comprises: a cloud transmission device 6 disposed in the drainage element disposing space 10 and coupled to the liquid detection device 5, wherein the cloud transmission device 6 is configured to transmit the liquid detection information from the liquid detection device 5 to a cloud data analyzing device D1 for cloud data analysis.

Specifically, the cloud transmission device 6 may transmit the liquid detection information to the cloud data analyzing device D1 via a wired or wireless network, thereby enabling the cloud data analyzing device D1 to perform a big data analysis and a various management based on the liquid detection information collected from multiple parties. The liquid detection information may include the liquid level information, the liquid color information, the tag identification information, or information related thereto or derived therefrom.

As shown in FIG. 2 and FIG. 5, according to one embodiment of the present invention, the drainage bottle system 100 further comprises: a wireless remote transmission device 7 disposed in the drainage element disposing space 10 and coupled to the liquid detection device 5, wherein the wireless remote transmission device 7 is configured to wirelessly transmit the liquid detection information from the liquid detection device 5 to a remote mobile device D2 for notification and/or display on the remote mobile device D2.

Specifically, by providing the wireless remote transmission device 7, it is convenient to transmit the liquid detection information to the remote mobile device D2 in a wireless manner such as Bluetooth and Wi-Fi so that the healthcare staff, the patient or patient's relatives can be informed of the relevant data immediately via the remote mobile device D2. Preferably, the remote mobile device D2 may be a wearable mobile device D3 such as a smart watch.

In addition, as shown in FIG. 2 and FIG. 5, according one embodiment of the present invention, the drainage bottle system 100 further comprises: a data port 8, which is, for example, a USB port for data transmission between the drainage bottle system 100 and an external device D4.

The above description should be considered as only the discussion of the preferred embodiments of the present invention. However, a person having ordinary skill in the art may make various modifications without deviating from the present invention. Those modifications still fall within the scope of the present invention.

What is claimed is:

1. A drainage bottle system, comprising:
a system housing having a drainage element disposing space;
a drainage bottle, mounted on the system housing;
a drainage tube, which is disposed within the system housing, having a sucking end extending outside the system housing and a collecting end connected to the drainage bottle through the system housing;
a motorless suction device, which is disposed in the drainage element disposing space, including a piezoelectric suction pump and a driving circuit, wherein the piezoelectric suction pump is connected with the drainage tube, the driving circuit is coupled to the piezoelectric suction pump and is configured to drive the piezoelectric suction pump so that the piezoelectric suction pump generates a negative pressure by means of piezoelectric effect to drive the drainage tube to suck a liquid to be detected from the sucking end toward the collecting end, thereby collecting the liquid to be detected via the drainage tube into the drainage bottle; and
a liquid detection device, disposed in the drainage element disposing space and corresponding to the drainage bottle to obtain at least one piece of liquid detection information about the liquid to be detected.

2. The drainage bottle system as claimed in claim 1, wherein the system housing is provided with a front outer surface as a drainage bottle mounting surface on which the drainage bottle is mounted, wherein the collecting end of the drainage tube extends through the drainage element disposing space to the drainage bottle mounting surface to be connected to the drainage bottle.

3. The drainage bottle system as claimed in claim 2, wherein the motorless suction device further includes a suction tube, one end of the suction tube is connected to the piezoelectric suction pump, the other end of the suction tube extends to the drainage bottle mounting surface to be connected to the drainage bottle, and thereby the piezoelectric suction pump is connected to the drainage tube via the suction tube and the drainage bottle, wherein the piezoelectric suction pump is configured to generate the negative pressure in the drainage bottle so that the liquid to be detected is sucked and collected into the drainage bottle via the drainage tube.

4. The drainage bottle system as claimed in claim 1, wherein the piezoelectric suction pump is a ceramic piezoelectric suction pump that utilizes mechanical deformation of a piezoelectric ceramic of the ceramic piezoelectric suction pump to generate the negative pressure.

5. The drainage bottle system as claimed in claim 1, wherein the driving circuit is configured to drive the piezoelectric suction pump with a sinusoidal wave signal or a square wave signal.

6. The drainage bottle system as claimed in claim 1, wherein the liquid detection device includes at least one of a liquid level sensor, a liquid color sensor and a tag image recognition sensor,
wherein the liquid level sensor is used for sensing a liquid level of the liquid to be detected in the drainage bottle to obtain liquid level information as the liquid detection information,
the liquid color sensor is used for sensing a liquid color of the liquid to be detected in the drainage bottle to obtain liquid color information as the liquid detection information, and
the tag image recognition sensor is used for correspondingly sensing a tag identification code on the drainage bottle to obtain tag identification information as the liquid detection information.

7. The drainage bottle system as claimed in claim 1, further comprising: a cloud transmission device disposed in the drainage element disposing space and coupled to the liquid detection device, wherein the cloud transmission device is configured to transmit the liquid detection information from the liquid detection device to a cloud data analyzing device for cloud data analysis.

8. The drainage bottle system as claimed in claim 6, further comprising: a cloud transmission device disposed in the drainage element disposing space and coupled to the liquid detection device, wherein the cloud transmission device is configured to transmit the liquid detection information from the liquid detection device to a cloud data analyzing device for cloud data analysis.

9. The drainage bottle system as claimed in claim 1, further comprising: a wireless remote transmission device disposed in the drainage element disposing space and coupled to the liquid detection device, wherein the wireless remote transmission device is configured to wirelessly transmit the liquid detection information from the liquid detection device to a remote mobile device for notification and/or display on the remote mobile device.

10. The drainage bottle system as claimed in claim 6, further comprising: a wireless remote transmission device disposed in the drainage element disposing space and coupled to the liquid detection device, wherein the wireless remote transmission device is configured to wirelessly transmit the liquid detection information from the liquid detection device to a remote mobile device for notification and/or display on the remote mobile device.

11. The drainage bottle system as claimed in claim 9, wherein the remote mobile device is a wearable mobile device.

12. The drainage bottle system as claimed in claim 10, wherein the remote mobile device is a wearable mobile device.
